(19) United States
(12) Patent Application Publication
(10) Pub. No.: US 2021/0386732 A1
(43) Pub. Date: Dec. 16, 2021

(54) SEMI-SOLID ANTI-HISTAMINE COMPOSITIONS AND METHODS OF MAKING AND USING THEREOF

(71) Applicant: SEATTLE GUMMY COMPANY, Seattle, WA (US)

(72) Inventors: Feng WAN, Issaquah, WA (US); William CARLSON, Shoreline, WA (US)

(21) Appl. No.: 16/963,228

(22) PCT Filed: Jan. 14, 2019
(86) PCT No.: PCT/US2019/013537

§ 371 Date: Jul. 19, 2020

Related U.S. Application Data
(60) Provisional application No. 62/617,303, filed on Jan. 15, 2018.
(60) Provisional application No. 62/683,523, filed on Jun. 11, 2018.

(51) Int. Cl. A61K 31/495; A61K 9/06; A61K 31/4545; A61K 31/55; A61K 31/40; A61K 31/445; A61K 31/451; A61K 31/5415; A61K 31/4458; A61K 31/554; A61K 47/69; A61K 47/26; A61K 36/28; A61K 36/87; A61K 36/534; A61K 36/484; A61K 35/748; A61K 36/752; A61K 36/16; A61K 36/258; A61K 36/537; A61K 36/53; A61K 31/355; A61K 31/375; A61K 31/522; A61K 31/047; A61K 33/04; A61K 36/064; A61K 36/185; A61K 31/05; A61K 36/88; A61K 31/015; A61K 31/07; A61K 31/593; A61K 31/122; A61K 33/06; A61K 33/26; A61K 33/30; A61K 33/14; A61K 33/34; A61K 33/24; A61K 33/32; A61K 33/42; A61K 33/18; A61K 31/4172; A61K 31/405; A61K 9/00

(52) U.S. Cl. CPC A61K 31/495; A61K 9/0056; A61K 31/4545; A61K 31/55; A61K 31/40; A61K 31/445; A61K 31/451; A61K 31/5415; A61K 31/4458; A61K 31/554; A61K 47/6951; A61K 47/26; A61K 36/28; A61K 36/87; A61K 36/534; A61K 36/484; A61K 35/748; A61K 36/752; A61K 36/16; A61K 36/258; A61K 36/537; A61K 36/53; A61K 31/355; A61K 31/375; A61K 31/522; A61K 31/047; A61K 33/04; A61K 36/064; A61K 36/185; A61K 31/05; A61K 36/88; A61K 31/015; A61K 31/07; A61K 31/593; A61K 31/122; A61K 33/06; A61K 33/26; A61K 33/30; A61K 33/14; A61K 33/34; A61K 33/24; A61K 33/32; A61K 33/42; A61K 33/18; A61K 31/4172; A61K 31/405; A61K 9/06

(57) ABSTRACT

The disclosure provides a semi-solid composition, comprising a gelling component in a sufficient amount to provide a cohesive gelled product, an anti-histamine composition, comprising an antihistamine, and a complexing agent, wherein the complexing agent is capable of interacting with the antihistamine and forming an antihistamine complex. In one embodiment, the antihistamine comprises diphenhydramine, cetirizine, levocetirizine, loratadine, desloratadine, fexofenadine, azelastine, bilastine, rupatadine, or a derivative, salt or a combination thereof.

### CROSS REFERENCE TO RELATED APPLICATIONS

This claims the benefit of the filing date of U.S. Provisional Application Ser. No. 62/617,303, filed Jan. 15, 2018, and U.S. Provisional Application Ser. No. 62/683,523, filed Jun. 11, 2018, the entire disclosures of which are incorporated by reference herein.

### TECHNICAL FIELD

This application relates to semi-solid edible or chewable compositions with one or more bioactive incorporated therein.

### BACKGROUND

Unless otherwise indicated herein, the materials described in this section are not prior art to the claims in this application and are not admitted being prior art by inclusion in this section.

Confectionery items make for excellent delivery materials for nutritionals such as vitamins and minerals. One confectionery form that is popular to deliver nutritional moieties is the gummy. A gummy is a chewable confectionery that is primarily made of a gelation agent, simple mono and disaccharides, and water. Popular gelling agents include pectin, starch and gelatin. Simple monosaccharides include glucose and disaccharides include sucrose. Sometimes a flavor is used with the gelling agents and sugars to increase the gummy composition's taste.

While gummy confectioneries have been popular for the delivery of vitamins, they have been not been used for the delivery of active pharmaceutical ingredient(s) (API). One reason is that many APIs are extremely foul or bitter in taste. The inclusion of the API in gummy format makes for a foul-tasting product that many would not want. Hence, pharmaceuticals have typically been reserved for non-chewable items that make for minimal mouth contact.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the semi-solid pharmaceutical composition comprises a gelling component in a sufficient amount to provide a cohesive gelled product, an anti-histamine composition and a complexing agent. The antihistamine composition includes an antihistamine. The complexing agent is capable of interacting with antihistamine and forming an antihistamine complex.

In one embodiment, the antihistamine composition comprises acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative thereof. In one embodiment, the antihistamine composition comprises diphenhydramine, cetirizine, levocetirizine, loratadine, desloratadine, fexofenadine, azelastine, bilastine, rupatadine, or a derivative, salt or a combination thereof. In one embodiment, the antihistamine is a H1-antihistamine. In one embodiment, the antihistamine comprises cetirizine, diphenhydramine, loratadine, or fexofenadine. In one embodiment, the antihistamine composition comprises cetirizine dihydrochloride or diphenhydramine dichloride.

In one embodiment, the antihistamine composition comprises essentially cetirizine. In one embodiment, the semi-solid pharmaceutical composition comprises cetirizine at a concentration not less than about 0.05%, 0.1%, or 0.2% w/w. In one embodiment, the semi-solid pharmaceutical composition of comprises cetirizine at a concentration of about 0.07% by weight. In one embodiment, the semi-solid pharmaceutical composition of comprises cetirizine at a concentration of about 0.14% by weight. In one embodiment, the semi-solid pharmaceutical composition of comprises cetirizine at a concentration of about 0.28% by weight.

In one embodiment, the antihistamine composition comprises essentially diphenhydramine. In one embodiment, the semi-solid pharmaceutical composition comprises diphenhydramine at a concentration not less than about 0.3%, 0.5%, or 1% w/w. In one embodiment, the semi-solid pharmaceutical composition comprises diphenhydramine at a concentration about 0.35% by weight. In one embodiment, the semi-solid pharmaceutical composition comprises diphenhydramine at a concentration about 0.71% by weight. In one embodiment, the semi-solid pharmaceutical composition comprises diphenhydramine at a concentration about 1.42% by weight.

In one embodiment, the antihistamine composition comprises essentially loratadine. In one embodiment, the semi-solid pharmaceutical composition comprises loratadine at a concentration not less than about 0.05%, 0.1%, or 0.2% w/w. In one embodiment, the semi-solid pharmaceutical composition of comprises loratadine at a concentration of about 0.07% by weight. In one embodiment, the semi-solid pharmaceutical composition of comprises loratadine at a concentration of about 0.14% by weight. In one embodiment, the semi-solid pharmaceutical composition of comprises loratadine at a concentration of about 0.28% by weight.

In one embodiment, the antihistamine composition comprises essentially fexofenadine. In one embodiment, the semi-solid pharmaceutical composition comprises fexofenadine at a concentration not less than about 0.1%, 0.2%, 0.3%, 0.4%, 0.6%, 0.8%, 1%, or 1.2% w/w. In one embodiment, the semi-solid pharmaceutical composition of comprises fexofenadine at a concentration of about 0.2% by weight. In one embodiment, the semi-solid pharmaceutical composition of comprises fexofenadine at a concentration of about 0.4% by weight. In one embodiment, the semi-solid pharmaceutical composition of comprises fexofenadine at a concentration of about 0.6% by weight.

The complexing agent is capable of complexing with the antihistamine through coordinating, chelating, complexing, hydrogen-bonding, dipole-dipole interaction, van-der waals interaction, or a combination thereof. In one embodiment, the antihistamine complex is capable of masking, lessening or reducing the antihistamine's taste, increasing antihistamine's solubility or stability in aqueous matrix, or a combination thereof. In one embodiment, the antihistamine complex is capable of masking and reducing the bitterness, astringent or metallic taste of the antihistamine. In one embodiment, the antihistamine complex is capable of increasing antihistamine's solubility in aqueous matrix therefore facilitating the incorporation of the antihistamine into the aqueous gummy matrix.

In one embodiment, the complexing agent comprises protein, peptide, amide or polyamide, cluster dextrin, cyclodextrin, polydextrose, resistant starch, polyethylene glycol, polyunsaturated hydrocarbons, polyunsaturated fatty acids, mica, talc, zeolite, cellulose, plant particles, calcium carbonate, diatomaceous earth, chitosan, or a combination thereof.

In one embodiment, the complexing agent comprises an amide. Example amide includes without limitation 2-deoxy-2-aminoglucose N-acetyl, sialic acid N-acetyl, iminosugar N-acetyl, daunosamine N-acetyl, 2-deoxy-2aminogalactose N-acetyl, chitin, pectin, and amino acids.

Plant particles may be derived from various parts of a plant such as flower, fruit, seed, grain, nut, nutshell, root, leaves, or stems. In one embodiment, the plant particles comprise berry powder, nutshell powder, rice bran powder including without limitation strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, cranberry powder or blueberry powder.

In one embodiment, the complexing agent comprises cluster dextrin or cyclodextrin. In one embodiment, the complexing agent comprises cyclodextrin. In one embodiment, the cyclodextrin comprises alpha-dextrin, beta-cyclodextrin, gamma-cyclodextrin, or a combination thereof. In one embodiment, the cyclodextrin comprises essentially gamma-cyclodextrin. In one embodiment, the semi-solid pharmaceutical composition comprises antihistamine and cyclodextrin at a molar ratio of from about 1:1 to about 1:100. In on embodiment, the molar ratio of antihistamine and cyclodextrin is from about 1:1 to about 1:20. In one embodiment, the molar ratio of antihistamine and cyclodextrin is about 1:5.

In one embodiment, the antihistamine composition comprises cetirizine, levocetirizine, diphenhydramine, loratadine, or fexofenadine, and the complexing agent comprises polyamide, cluster dextrin, cyclodextrin, or a combination thereof. In one embodiment, the antihistamine composition comprises cetirizine and the complexing agent comprises alpha-cyclodextrin. In one embodiment, the antihistamine composition comprises cetirizine and the complexing agent comprises beta-cyclodextrin. In one embodiment, the antihistamine composition comprises cetirizine and the complexing agent comprises gamma-cyclodextrin. In one embodiment, the antihistamine composition comprises diphenhydramine and the complexing agent comprises cyclodextrin, cluster dextrin, or a combination thereof. In one embodiment, the antihistamine composition comprises loratadine and the complexing agent comprises cyclodextrin.

The gelling composition may include gelatin, starch, pectin, gellan gum, gum Arabic, carrageenan, guar, agar, alginate, locust bean gum, xanthan, or derivatives thereof. In one embodiment, the gelling composition comprises pectin and gelatin in a ratio from about 10:1 to about 1:1. In one embodiment, the gelling composition comprises gelatin and starch in a ratio from about 100:1 to about 1:100. In one embodiment, the gelling composition consists essentially of starch, gelatin, alginate or pectin. In one embodiment, the gelling composition consists essentially of gelatin.

In one embodiment, the gelling component consists essentially of pectin. In one embodiment, the gelling component comprises apple pectin, citrus pectin, or a combination thereof. In one embodiment, the semi-solid composition comprises at least 1% of pectin. In one embodiment, the semi-solid composition comprises from about 1% to about 5% of pectin. In one embodiment, the semi-solid composition comprises about 2.5% pectin. In one embodiment, pectin has a methoxy content of not less than 30%, 40% or 50%. In one embodiment, pectin has an amid content of not less than 10%, 15%, 20%, 25%, 30% or 40%. In one embodiment, pectin has a carboxylic content of not less than 25%, 30%, 35%, 40%, 50%, or 60%. In one embodiment, the pectin has a methyl ester not more than 30%, 32%, 35%, or 40%.

The semi-solid pharmaceutical composition may further comprise an herb composition, an antioxidant composition, a vitamin composition, a mineral composition, an amino acid composition, a probiotics composition, or a prebiotics composition. The herb composition, the antioxidant composition, the vitamin composition, the mineral composition, the amino acid composition, the probiotics composition or the prebiotics composition may work synergistically with the antihistamine helping to relieve the allergy or the symptoms of allergy (such as inflammation, hives, congestion, secretions, and other respiratory symptoms).

In one embodiment, the herb composition comprises one or more herbs having biological activity for alleviating or soothing allergy symptoms. In one embodiment, the herb composition works with the antihistamine synergistically therefor is configured to enhance the antihistamine activity or increase the anti-allergy activity of the pharmaceutical composition. In one embodiment, the herb composition may have the antihistamine activity. In one embodiment, the herb composition comprises butterbur, quercetin, Stinging nettles (Urtica dioica), bromelain, phleum pratense, tinospora cordifolia, European elderflower, sorrel, cowslip, verbena, gentian root, echinacea, grape seed, pycnogenol, pine bark extract, EPA, honey, cat's claw, albizzia (Albizzia lebbeck), baical skullcup (Scutellaria baicalensis), goldenseal, spirulina, bitter orange (citrus aurantium), lemon, eucalyptus, frankincense, Angelica sinensis, eyebright (Euphrasia officinalis), Gingko, milk thistle (Silybum marianum), red clover (Trifolium pratense), Yarrow (Achillea millefolium), rosemary, shiso, sage, peppermint, turmeric, licorice, Astragalus, Ginseng, Artemisia argyi, Stephania tetrandra, coix seed, Citrus trifoliata, Citrus aurantium, Angelica dahurica, an extract, isolate or distillate thereof.

In one embodiment, the herb composition comprises butterbur, its extract or powder thereof. In one embodiment, the herb composition comprises stinging nettle leaf, its extract or powder thereof. In one embodiment, the herb composition comprises turmeric, its extract or powder thereof. In one embodiment, the herb composition comprises angelica sinesis, its extract or powder thereof. In one embodiment, the herb composition comprises milk thistle (Silybum marianum), its extract or powder thereof.

In one embodiment, the antioxidant composition comprises Vitamin E, Vitamin C, beta-carotene, gallic acid, selenium, selenium yeast, phenolics, anthocyanins, flavonoids, bioflavonoids, theobromine, anthracenes, carotenoids, lutein, zeaxanthin, gingko biloba, blackberry extract, elderberry extract, cranberry extract, blueberry extract, grapeseed extract, resveratrol, saffron, Sangre de grado (dragon's blood), cocoa, or derivatives thereof. In one embodiment, the vitamin composition comprises vitamin A, B, C, D, E, K or a combination thereof. In one embodiment, vitamin B comprises thiamin (B1), riboflavin (B2), niacin or niacinamide (B3), pantothenic acid (B5), pyridoxines (B6), biotin (B7), folate or folic acid (B9), cobalmine (B12), or their derivative thereof. In one embodiment, the vitamin composition consists essentially of vitamin C or the derivative thereof.

The mineral composition may have the biological activity for alleviating or soothing allergy symptoms. In one embodiment, the mineral composition may work with the antihistamine synergistically therefor is configured to enhance the antihistamine activity or increase the anti-allergy activity of the pharmaceutical composition. In one embodiment, the mineral composition comprises salts of calcium, iron, zinc, magnesium, sodium, chloride, potassium, copper, molybdenum, manganese, phosphorus, iodine, nickel, or selenium, or a combination thereof. In one embodiment, the mineral composition consists essentially salts of zinc.

In one embodiment, the amino acid composition comprises one or more amino acids having biological activity for alleviating or soothing allergy symptoms. In one embodiment, the amino acid composition works with the antihistamine synergistically therefor is configured to enhance the antihistamine activity or increase the anti-allergy activity of the pharmaceutical composition. In one embodiment, the amino acid composition may have the antihistamine activity. In one embodiment, the amino acid composition comprises histidine, a branched chain amino acid, L-5 hydroxytryptophan (5-HTP), or its derivative thereof.

In one embodiment, the prebiotic composition comprises gum arabic, chicory root, wheat bran, resistant starch, mannose oligosaccharide, acacia gum, inulin, galacto-oligosaccahride, guar gum, Artichoke fiber, fructo-ligosaccharide, or a combination thereof.

The probiotic composition comprises bifidobacteria, lactic acid bacteria, or a combination thereof. In one embodiment, the probiotic composition comprises Bifidobacterium lactis, Bifidobacterium longum, Lactobacillus acidophilus, Lactobacillus paracasei, Lactobacillus plantarum, Lactobacillus rhamnosus, Bacillus coagulans, Bifidobacterium bifidum, Lactobaccillus casei, Lactobaccillus gasseri, Lactobacillus salivarius, Lactobacillus bulgarius, or a combination thereof.

The semi-solid pharmaceutical composition may further comprise an additive selected from sweeteners, food acids, flavoring agents, coloring agents, humectants, bulking agents, fatty acids, triglycerides, plasticizers, emulsifiers, thickeners, preservatives, or and a mixture thereof. In one embodiment, the sweetener comprises erythritol, xylitol, sugar, glucose syrup, corn syrup, high fructose corn syrup, trulinose, trehalose, isomaltose, psicose, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

In one embodiment, the flavoring agent comprises vanilla, chili oil, gingerol, peperine, capsaicin, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, bitter orange flavor, fruit essences, Rosemary Oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, cocoa, pineapple flavor, or mixtures or derivatives thereof.

In one embodiment, the semi-solid pharmaceutical composition may include a sugar composition. The sugar composition may function as a sweetener, as a bonding agent assisting the gelation of the gelling component, or a combination thereof. The sugar composition may include high glycemic index sugars. In one embodiment, the high glycemic index sugar has a glycemic index of greater than 50, 60, 70, 80, 90, 100, 120, 150 or 160. Examples high glycemic index sugar include without limitation sucrose, glucose, dextrin, maltose, maltotriose, or maltodextrin. In one embodiment, the sugar composition comprises sucrose, glucose or a combination thereof.

In one embodiment, the sugar composition may include low glycemic sugars. In one embodiment, the low glycemic sugar has a glycemic index less than 50, 40, 30, 25, 20, 15, or 10. Examples of low glycemic index sugar include without limitation trehalose, palatinose, isomaltulose, tagatose, sorbose, galactose, mannose, psicose, or fructose. In one embodiment, the sugar composition comprises trehalose, palatinose (isomaltulose), psicose (allulose), or a combination thereof. In one embodiment, the sugar composition comprises trehalose and palatinose. In one embodiment, the ratio of trehalose and palatinose is from about 10:1 to 1:10. In one embodiment, the sugar composition comprises palatinose and psicose. In one embodiment, the ratio of palatinose and psicose is from about 1:10 to about 10:1. In one embodiment, the sugar composition comprises trehalose and psicose. In one embodiment, ratio of trehalose and psicose is from about 1:10 to about 10:1. In one embodiment, the sugar composition comprises trehalose, palatinose, and psicose. The ratio of various sugars may be any ratio in between the ranges.

The semi-solid pharmaceutical composition may have a glycemic index from about 8 to about 170. In one embodiment, the semi-solid pharmaceutical composition has a glycemic index of more than 60. In one embodiment, the semi-solid pharmaceutical composition has a glycemic index of more than 90. In one embodiment, the semi-solid pharmaceutical composition may have a glycemic index from about 50 to about 170. In one embodiment, the semi-solid pharmaceutical composition may have a glycemic index from about 50 to about 80. In one embodiment, the semi-solid pharmaceutical composition has a glycemic index of not more than 30. In one embodiment, the semi-solid pharmaceutical composition has a glycemic index of not more than about 20. In one embodiment, the semi-solid pharmaceutical composition ha a glycemic index of not more than about 15. In one embodiment, the semi-solid pharmaceutical composition ha a glycemic index from about 8 to about 25.

The semi-solid pharmaceutical composition may have a pH from about 2 to about 6. In one embodiment, the pH of the composition is about 2.5 to about 2.9. In one embodiment, the pH of the composition is about 2.7 to about 2.9. In one embodiment, the pH of the composition is about 2.6 to about 3. In one embodiment, the pH of the composition is about 3 to about 5. In one embodiment, the pH of the composition is about 3.0 to about 3.4. The pH of the composition may be any number in between the ranges.

In one embodiment, the application provides a semi-solid pharmaceutical composition, comprising pectin from about 0.5% to about 5.0% by weight, cetirizine from about 0.05% to about 0.3% by weight, a complexing agent from about 0.1% to about 12% by weight, and a sugar composition from about 50% to about 85% by weight. In one embodiment, the complexing agent comprises polyamide, cyclodextrin, or cluster dextrin. In one embodiment, the complexing agent comprising essentially cyclodextrin. In one embodiment, the pharmaceutical composition has a glycemic index of more than 80. In one embodiment, the pharmaceutical composition has a glycemic index of less than 30. In one embodiment, the pharmaceutical composition has a glycemic index from about 8 to about 25.

In one embodiment, the application provides a semi-solid pharmaceutical composition, comprising pectin from about 0.5% to about 5.0% by weight, diphenhydramine from about 0.3% to about 1.5% by weight, a complexing agent from about 0.1% to about 12% by weight, and a sugar composition from about 50% to about 85% by weight. In one embodiment, the complexing agent comprises polyamide, cyclodextrin, or cluster dextrin. In one embodiment, the complexing agent comprising essentially cyclodextrin. In one embodiment, the pharmaceutical composition has a glycemic index of more than 70. In one embodiment, the pharmaceutical composition has a glycemic index of less than 25. In one embodiment, the pharmaceutical composition has a glycemic index from about 8 to about 25.

In one embodiment, the application provides a semi-solid pharmaceutical composition, comprising pectin from about 0.5% to about 5.0% by weight, loratadine from about 0.05% to about 0.3% by weight, a complexing agent from about 0.1% to about 12% by weight, and a sugar composition from about 50% to about 85% by weight. In one embodiment, the complexing agent comprises polyamide, cyclodextrin, or cluster dextrin. In one embodiment, the complexing agent comprising essentially cyclodextrin. In one embodiment, the pharmaceutical composition has a glycemic index of more than 70. In one embodiment, the pharmaceutical composition has a glycemic index of less than 25. In one embodiment, the pharmaceutical composition has a glycemic index from about 8 to about 25.

In one embodiment, the application provides a semi-solid pharmaceutical composition, comprising pectin from about 0.5% to about 5.0% by weight, fexofenadine from about 0.1% to about 1% by weight, a complexing agent from about 0.1% to about 12% by weight, and a sugar composition from about 50% to about 85% by weight. In one embodiment, the complexing agent comprises polyamide, cyclodextrin, or cluster dextrin. In one embodiment, the complexing agent comprising essentially cyclodextrin. In one embodiment, the pharmaceutical composition has a glycemic index of more than 80. In one embodiment, the pharmaceutical composition has a glycemic index of less than 30. In one embodiment, the pharmaceutical composition has a glycemic index from about 8 to about 25.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the chemical structure of example antihistamine, cetirizine (both L and D isomers) and diphenhydramine;

FIG. 2 shows that trehalose digests and releases glucose slowly when compared to other food providing a low glycemic value;

FIG. 3 shows that palatinose digests very slowly and has low impact on blood glucose levels providing a low glycemic value;

FIG. 4 shows the simplified structure of cluster dextrins, which have a ring structure with pendent chains of glucose;

FIG. 5 shows the ring structures of alpha, beta, and gamma cyclodextrin;

FIG. 6 shows the ring structure of the cyclodextrin complexing with cetirizine molecule to form an inclusion complex;

FIG. 7 shows the chromatograph and mass spectrometry of the representative pharmaceutical gummy composition from Example 12, in which the protonated form of cetirizine eluted at 4:50 and 4:63 for the D and L stereoisomers, indicating that cetirizine is stable in the representative gummy formulation;

FIG. 8 is the chromatograph and mass spectrometry of the gummy sample from Example 12, showing that cetirizine is stable in the representative gummy formulation;

FIG. 9 shows that liquid chromatography mass spectrometry of the representative pharmaceutical gummy composition yielded in Example 29, in which cetirizine molecule eluted at 4.38 indicating that cetirizine is stable in the representative gummy formulation; and

FIG. 10 shows the liquid chromatography mass spectrometry of the representative pharmaceutical gummy composition yielded in Example 44, in which the protonated form of the diphenhydramine molecule eluted at 3.79 indicating that diphenhydramine is stable in the representative gummy formulation.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

Semi-solid formulation, such as gummy, can be used as an efficient delivery mechanism for an active pharmaceutical ingredient (API). A good tasting pharmaceutical gummy composition would have several advantages over traditional pharmaceutical delivery formulations such as tablets, capsules and syrups. Unlike tablets and capsules, gummy can be taken with population with swallowing issues such as children and seniors. Unlike syrups, gummy can be accurately dosed. The pharmaceutical gummy composition that is sweet and pleasing to consume leads to the advantage of increased patients' compliance in taking the medication. The act of chewing and dissolving the gummy allows for releasing of the API in the mouth and trans-mucosal absorption of the API. The rapid absorption of the API allows for the API to bypass the liver and avoid first-pass effect. Furthermore, trans-mucosal absorption allowed by the gummy gives faster symptom relief due to fast uptake of the API. Thus, solving the problems with gummy delivery of API's can lead to a superior technology for API delivery.

However, most of the APIs (such as cetirizine, diphenhydramine, etc.) have bitter, astringent, metallic or foul taste and are unpleasing. When placed into a traditional confectionary gummy formulation, the foul taste such as bitterness and metallic taste stays regardless of the quantity of sweeteners used. While a gummy delivery of APIs such antihistamines would be advantageous due to rapid absorption and symptom relief, simply incorporating these APIs into traditional gummy confectionary only lead to foul tasting undesirable products. Furthermore, gummy formulation is water based providing an aqueous matrix. Usually, a gummy formulation contains from about 12% to about 20% by weight of water content. APIs tend to instable in such aqueous environment. In addition, APIs are usually not water soluble make it technically impossible to disperse APIs homogenously into an aqueous gummy matrix to produce consistent products.

This application solved the above technical problems by providing semi-solid pharmaceutical gummy formulations for the delivery APIs with pleasing taste and stability profile as well as homogenous products. In some embodiments, the application provides gummy pharmaceutical formulations that is excellent in texture, taste, and flavor, with proven solubility and stability of the APIs and allows for rapid delivery of antihistamines for fast relief of allergy symptoms.

The semi-solid pharmaceutical gummy composition may include an anti-histamine composition comprising an antihistamine. Example antihistamines may include acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative thereof. The anti-histamine composition may include one or more antihistamines.

In one embodiment, the antihistamine composition may include diphenhydramine, cetirizine, levocetirizine, loratadine, desloratadine, fexofenadine, azelastine, bilastine, rupatadine, or a derivative, salt or a combination thereof. In one embodiment, the antihistamine comprises cetirizine. In one embodiment, the antihistamine comprises diphenhydramine.

Both cetirizine and diphenhydramine are API's that act as an antihistamine. Both cetirizine and diphenhydramine used herein include all pharmaceutically useful derivative forms such as salts. In one embodiment, cetirizine may be cetirizine hydrochloride salt. In one embodiment, diphenhydramine may be diphenhydramine hydrochloride salt.

Cetirizine and diphenhydramine are indicated for symptom relief from allergies and colds. Cetirizine has a much lower indication of drowsiness side effect. In addition to relieving allergy symptoms, diphenhydramine can help with nausea and provide relief for motion sickness. The drowsiness causing effect makes diphenhydramine an effective sleep aid.

The pharmaceutical gummy delivers from about 1 mg to about 80 mg of antihistamine APIs per dosage. In one embodiment, the pharmaceutical gummy delivers at least 2 mg cetirizine hydrochloride per dose. In one embodiment, the pharmaceutical gummy delivers 2.5 mg, 5 mg, or 10 mg of cetirizine hydrochloride per dose.

In one embodiment, the pharmaceutical gummy delivers at least 8 mg of diphenhydramine hydrochloride per dose. In one embodiment, the pharmaceutical gummy delivers 12.5 mg, 25 mg, or 50 mg of diphenhydramine hydrochloride per dose.

In one embodiment, the pharmaceutical gummy delivers at least 2 mg loratadine per dose. In one embodiment, the pharmaceutical gummy delivers 2.5 mg, 5 mg, or 10 mg of loratadine per dose.

In one embodiment, the pharmaceutical gummy delivers at least 15 mg fexofenadine per dose. In one embodiment, the pharmaceutical gummy delivers 15 mg, 30 mg, 60 mg, 90 mg, or 180 mg of fexofenadine per dose.

The pharmaceutical gummy may weight from about 2 g to about 10 g per dose. In one embodiment, the pharmaceutical gummy weights about 2.5 g, 3 g, 3.5 g, 4 g, 5 g, 6 g, 6.5 g, 7 g, or 7.5 g per dose. The weight per dose may be any number in between the ranges.

The pharmaceutical gummy may include a sugar composition. In one embodiment, the sugar composition may act as a sweetener, a bonding agent for the gelling component, or both. In one embodiment, the pharmaceutical gummy composition comprises not less than 60% of the sugar composition. In one embodiment, the pharmaceutical gummy composition may include from about 40% to about 85% the sugar composition. In one embodiment, the pharmaceutical gummy composition may include about 75% of sugar composition.

In one embodiment, the sugar composition may include sucrose, glucose, fructose, maltose, mannose, trehalose, palatinose, psicose, sorbose, tagatose, galactose, lactose, tagatose, sorbose, galactose, maltotriose, maltodextrin, glucosamine, N-acetylglucosamine, N-acetylgalactosamine, or a combination or derivative thereof. In one embodiment, the sugar composition may include sucrose, glucose, fructose, or a combination thereof. In one embodiment, the pharmaceutical gummy composition comprises from about 0.1% to about 55% of mannose. In one embodiment, the pharmaceutical gummy composition comprises from about 0.1% to about 85% of sucrose. In one embodiment, the pharmaceutical gummy composition comprises from about 0.1% to about 85% of fructose.

In one embodiment, the sugar composition comprises a low glycemic sugar. In one embodiment, the low glycemic sugar has a glycemic value of not more than 8, 10, 15, 20, 25, 30, or 35. Examples of the low glycemic sugars include, without limitation, L-glucose, L-sucrose, L-galactose, D- or L-isomaltulose, D or L-trehalose, D- or L-psicose, D- or L-tagatose, and D- or L-sorbose. In one embodiment, the sugar composition may include trehalose, palatinose, psicose, tagatose, sorbose, or a combination thereof. In some embodiments, the gummy compositions comprise not less than 68% weight of low glycemic sugars. In some embodiment, the gummy compositions comprise from about 45% weight to about 85% of low glycemic sugar.

In one embodiment, the pharmaceutical gummy composition comprises from about 50% to about 85% of the sugar composition. In one embodiment, the pharmaceutical gummy composition comprises from about 5% to about 55% of trehalose. In one embodiment, the pharmaceutical gummy composition comprises from about 5% to about 55% of palatinose. In one embodiment, the pharmaceutical gummy composition comprises from about 15% to about 75% of psicose. In one embodiment, the pharmaceutical gummy composition comprises from about 5% to about 75% of sorbose. In one embodiment, the pharmaceutical gummy composition comprises from about 5% to about 65% of tagatose.

In one embodiment, the pharmaceutical gummy composition comprises psicose and trehalose at a ratio from about 1:10 to about 10:1. In one embodiment, the pharmaceutical gummy composition comprises trehalose and palatinsoe at a ratio from about 1:10 to about 10:1. In one embodiment, the pharmaceutical gummy composition comprises palatinose and trehalose at a ratio from about 1:10 to about 10:1. The ratio may be any number in between the ranges such as 8:1 to 1:8, 1:5 to 5:1, 4:1 to 1:4, 3:1 to 1:3, 1:2 to 2:1, or 1:1.

In one embodiment, the pharmaceutical gummy composition may be substantially free of sucrose, fructose, glucose, or a combination thereof. In one embodiment, the gummy composition may be substantially free of sugar substitutes. In one embodiment, the gummy composition may be substantially free of artificial sweeteners. In one embodiment, the gummy composition may be substantially free of sugar alcohols.

In one embodiment, the pharmaceutical gummy may be a sugar free composition. In one embodiment, the pharmaceutical gummy may include a sugar alcohol composition. In one embodiment, the sugar alcohol composition may act as a sweetener, a bonding agent for assisting the gelation of the gelling component, or both. Examples sugar alcohol may include glycerol, sorbitol, mannitol, xylitol, or erythritol. In one embodiment, the pharmaceutical gummy may be gelatin-based gummy with a sugar substitute (such as stevia) as sweetener leading to a sugar free formulation.

The pharmaceutical gummy may include a food acid composition. In one embodiment, the food acid composition may act to impart acidic or astringent flavor, facilitate the gelling of the gelling component, or both. In one embodiment, the food acid composition may include citric acid, malic acid, ascorbic acid, lactic acid, galactic acid, glutamic acid, tartaric acid, propionic acid, butyric acid, valeric acid, gluconic acid, isocitric acid, succinic acid, fumaric acid or a combination there. In one embodiment, the food acid composition may include citric acid, malic acid, or a combination there.

The pharmaceutical gummy may include a buffer composition. The buffer composition acts to buffer the pH of the composition when combined with one of the above acid examples, facilitate the gelling, or both. In one embodiment, the buffer composition comprises sodium citrate, potassium citrate, calcium citrate, sodium hydroxide, potassium hydroxide, calcium hydroxide, sodium carbonate, sodium bicarbonate, potassium carbonate, potassium bicarbonate, a combination thereof.

In one embodiment, the gummy pharmaceutical formulation includes a gelling component, a sugar composition, water, and a complexing component. The complexing component complexes with antihistamine to form an inclusion complex. When APIs such as cetirizine or diphenhydramine molecules are coordinated within an inclusion complex, the bitter taste is reduced or eliminated, leading to a sweet, flavorful and pleasing pharmaceutical gummy.

In one embodiment, the pharmaceutical gummy formulation includes a gelling component, a sugar composition, and a food acid composition, flavor, and colorant. In one embodiment, the gelling component comprises gelatin or pectin. In one embodiment, the sugar composition comprises sucrose, fructose, glucose, trehalose, palatinose, or isomaltuose. In one embodiment, the food acid composition comprises citric acid, malic acid, or any combination thereof.

The pharmaceutical gummy formulation includes a complexing agent that is configured to complex with antihistamines. In one embodiment, the complexing agent may be a molecule made of glucose units arranged in ring or crown structure. Without limited by the theory, in some embodiments, the cetirizine molecule or diphenhydramine molecule may fit inside the ring or crown structure, at least partially, providing an inclusion complex. The formation of the inclusion complex greatly reduces the bitterness of the resulting API containing gummy pharmaceutical formulations.

### Cetirizine and Diphenhydramine

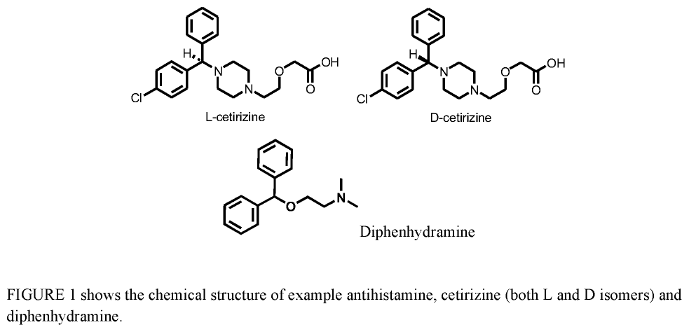

The chemical structures for cetirizine and diphenhydramine are shown in FIG. 1. The cetirizine structure is based upon an interior piperazine moiety. This moiety contributes to the bitterness of the molecule as the nitrogen atoms are strongly alkaline. The structure has both right handed (D) and left handed (L) stereo isomers. The L stereo isomer of cetirizine is marketed as levocetirizine. The L stereoisomer of cetirizine is the more biologically active of the two isomers. Diphenhydramine has similar structural elements in common with cetirizine. These structural elements would include a diphenyl moiety and tertiary amines. Both antihistamines function by acting primarily as an inverse agonist of the histamine H1 receptor.

Cetirizine and diphenhydramine are indicated for allergies that often affect the nose, sinuses, throat, and other areas of upper respiratory system. Both antihistamines act to relieve mild to moderate allergy symptoms including without limitation sneezing, runny nose, itchy or watery eyes and itchy throat or nose.

Antihistamine may also help relieve skin hives. Hives often occur with food or medication allergies. Unlike other antihistamine drugs, cetirizine crosses the blood-brain barrier only slightly and as such cetirizine produces minimal sedation compared to many other antihistamines for this reason. Diphenhydramine, on the other hand, is a smaller molecule that can easily cross the blood-brain barrier and can cause drowsiness when taken. Diphenhydramine is also indicated for treating nausea, motion sickness relief and as a sleep aid.

### Semi-Solid Chewable Gummy Formulation

### Gelling Agents

A large part of the texture of gummy products comes from the gelling agents used. The gelling agents are typically high molecular weight polymers.

Pectin is a heteropolysaccharide consisting of galactogluconic acid found in plant cell walls. Traditional pectin gels in the presence of sugar and acid, while chemically modified pectin gels in the presence of calcium or potassium ions instead of acid and is thus effective in low-acid foods. The latter will also work with lower solid concentrations. Pectin is water soluble. Pectin may be used in combination with gelatin. Gelatin provides a tough chewiness, while pectin lends a distinct tenderness.

Gelatin is a protein made from animal collagen. The source of collagen is often derived from cattle and pigs, but sometimes fish. Gelatin may be combined with other hydrocolloids-pectin, agar, starch, gum arabic—to create desired textures. In one embodiment, gelatin may be combined with gum arabic as the gelling agent.

In one embodiment, the gelling composition comprises starch. In one embodiment, the starch comprises amylose starch. For gummy formulations, the starch is often “modified”. There are a variety of modification techniques but common ones are contacting starch with acid, sodium or potassium hydroxide, or oxidizing the starch. These treatments help the starch to dissolve in water and gel to an appropriate level.

Agar or agar-agar is a jelly-like substance, obtained from algae. Agar is derived from the polysaccharide agarose and contains two components: the linear polysaccharide agarose, and a heterogeneous mixture of smaller molecules called agaropectin. Agar gels tend to weep or extrude water over time when used by itself as a gelling agent. In one embodiment, agar is combined with locust bean gum as a gelling agent. Locust bean gum helps to prevent weeping of agar gels. Locust bean gum is a galactomannan polysaccharide vegetable gum extracted from the seeds of the carob tree. The two polysaccharides from agar and locust bean gum synergistically interact with each other to form a strong gel that does not weep.

Carrageenans or carrageenins are a family of linear sulfated polysaccharides that are extracted from red edible seaweeds. The linear saccharide chains have a tendency to curl to form helical structures. Kappa-carrageenan has one sulphate group per disaccharide and forms strong, rigid gels in the presence of potassium ions. Similar to agar, locust bean gum is often used with kappa-carrageenan to prevent water from being expelled from the bulk of the gel (weeping). Gels formed from kappa-carrageenan and potassium ions are thermally reversible, meaning they melt when heated and solidify when cooled.

Alginic acid is a linear copolymer with homopolymeric blocks of (1-4)-linked β-D-mannuronate (M) (acid form of mannose) and its C-5 epimer α-L-guluronate (G) (acid form of gulose) residues, respectively, covalently linked together in different sequences or blocks. The monomers can appear in homopolymeric blocks of consecutive G-residues (G-blocks), consecutive M-residues (M-blocks) or alternating M and G-residues (MG-blocks). Alginate forms strong hydrogels when crosslinked with calcium ions.

### Simple Carbohydrates

Carbohydrates are an important constituent of the gummy structure. The carbohydrates help binds the gummy together through interaction with the gelling agent. The carbohydrates keep the texture of the product in a soft gum like state by acting as a humectant. The carbohydrates bind water. By binding water, the carbohydrates prevent the product from crystalizing, from drying out, and giving the product a chewy texture.

Sucrose, commonly known as table sugar, is a disaccharide consisting of one glucose unit and one fructose unit. The IUPAC name is O-α-D-glucopyranosyl-(1→2)-β-D-fructofuranoside. Sucrose is the most common carbohydrate used for gummy confectioneries. Sucrose is extremely soluble in water. More than 2 grams of sucrose will dissolve into 1 gram of water. The interaction between sucrose and water allows for sucrose to serve as a humectant for the confectionery. Sucrose helps the gummy to retain water and maintain its texture for longer periods of time. Sucrose also provides for sweetness of the product. Sucrose is the starting material for invert sugar. Invert sugar is sucrose that has undergone hydrolysis to yield the glucose and fructose monosaccharides. Invert sugar may be used for gummy formulation. Fructose, known as fruit sugar, is a monosaccharide, a ketose. Fructose is also very water soluble. Nearly 4 grams of fructose will dissolve into one gram of water. Due to the interaction between fructose and water it is used as a humectant in gummy formulation. Fructose helps gummy composition retain water, maintain texture, and prevent crystallization. Fructose has twice the sweetness of sucrose and is often used to increase the sweetness

Glucose is an aldose (a sugar with an aldehyde group or CHO) in its straight chain form. However, only 0.25% of glucose molecules exist in the straight chain form. Glucose performs an internal cyclization as shown below to yield α-D-glucopyranose and β-D-glucopyranose. In gummies, glucose is used to maintain the texture of the gummy and preventing crystallization of the sugars from gummy.

Psicose, sorbose, and tagatose are sugars similar to fructose. Chemically, they are ketoses and are C3-C5 stereoisomers of fructose. Psicose is identical to sucrose (table sugar) in sweetness but has nearly zero calories and does not promote tooth decay. Tagatose is nearly a sweet as sucrose yet only has 38% of the caloric value of sucrose and is much more tooth friendly than sucrose. Sorbose is used in the manufacture of vitamin C and is equivalent to sucrose in sweetness. Through extensive experimentation, processes are developed to used Psicose, sorbose, or tagatose in the semi-solid gummy formulation allowing these sugars behave like sucrose and fructose but without the caloric significance of sucrose.

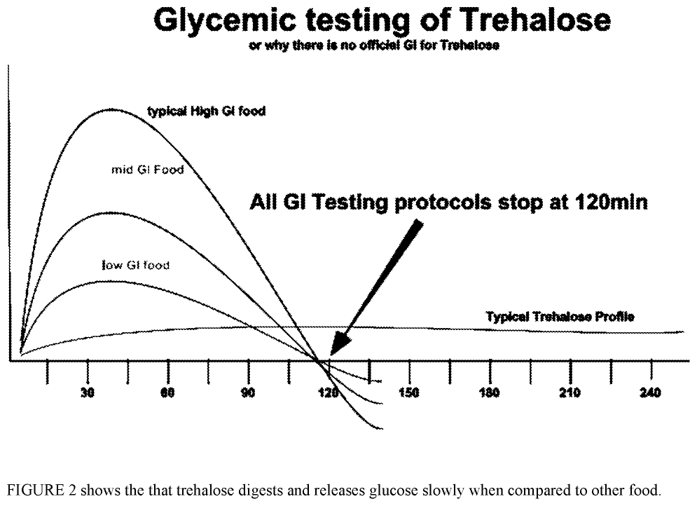

Trehalose, also known as mycose or tremalose, is a natural alpha-linked disaccharide formed by an α,α-1,1-glucoside bond between two α-glucose units. Trehalose is a non-reducing. Trehalose is reported to have antioxidant effects. Trehalose is also reported to have many significant neurological benefits. Trehalose is digested in the small intestine by the enzyme trehalase to release two molecules of glucose. The glucose molecules are then used by the body of the animal for energy. However, the digestion of trehalose does not lead to a spike in glucose levels in the blood, as shown in FIG. 2. Rather, the glucose levels slowly rise and are sustained over a longer period of time. Furthermore, there is no depletion of glucose over the long term, which is very unlike high glycemic index sugars. This is due to the digestion of trehalose in the small intestine rather than the mouth, trehalose has a much lower instance of causing dental decay than most carbohydrates, which is beneficial for the delivery of medications.

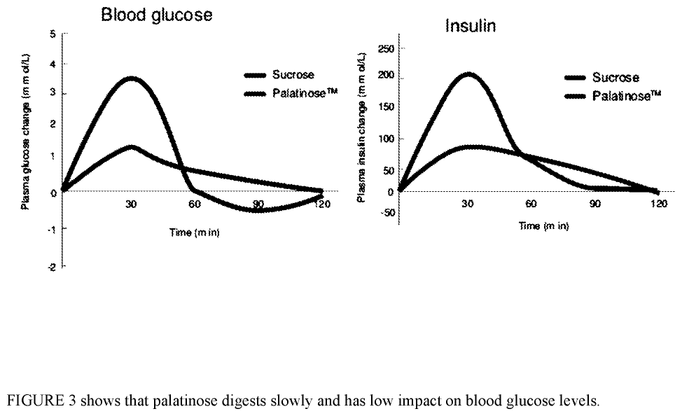

Palatinose, also known as isomaltulose, is a derivative of a natural source of sucrose. Palatinose is made by enzymatic rearrangement of the alpha-1,2 bond between the glucose and the fructose molecule to an alpha-1,6 bond. Palatinose is digested by enzymatic action by the enzyme sucrase. However, due to the rearrangement of palantinose vs. sucrose, sucrase hydrolysis of palatinose is much slower. The products of the hydrolysis are glucose and fructose. The glucose can be directly used by the body for energy while the fructose is converted to glucose by the liver. Due to its slow and full absorption and hydrolysis, palatinose supplies constant and long-lasting energy to the muscles and brain. Palatinose is slowly metabolized by the body causing only a minor increase in blood glucose level, as it enters the blood slowly. The result being that the energy obtained from Palatinose remains in the body much longer and offers a constant supply over a longer period than e.g. glucose or sucrose. Palatinose has limited influence on the blood glucose level and a low insulin index (FIG. 3). Palatinose has a low insulin index (about 30) and therefore may help to stabilize the insulin level in our body. Palatinose also has lower instances of tooth decay.

### Polydextrose

Polymers of glucose can exist in a variety of forms. Some of the forms of polymeric glucose are cyclic ring structures. The cyclic polymers of glucose can exist in several different forms. The ring structures can be highly branched and are often called cluster dextrins.

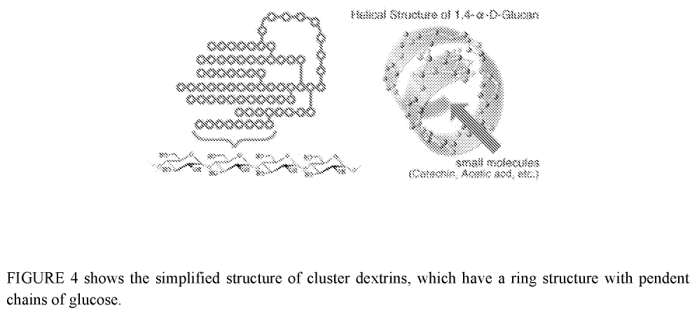

FIG. 4 shows the simplified structure of cluster dextrins. Cluster dextrins have a ring structure with branches of long chains of glucose units pendent to the ring. This has the effect of forming a helical structure. The helical structure along with the ring structure of cluster dextrins are both able to complex APIs in the current application. The helical structure along with the ring structure of cluster dextrin are both able to complex cetirizine and diphenhydramine molecules. For cetirizine, the complexing takes place by the phenyl groups on cetirizine fitting inside the helical structure.

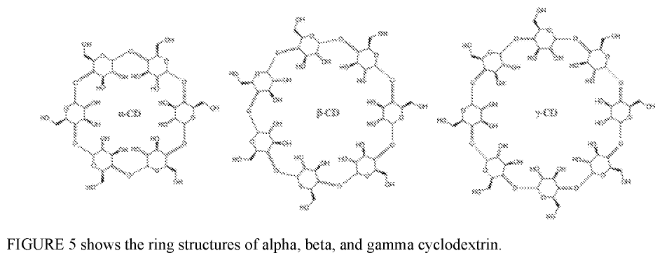

Cyclic dextrin is the other form of ring structured glucose. There are three major forms of cyclodextrin: alpha, beta, and gamma. FIG. 5 shows the ring structures of alpha, beta, and gamma cyclodextrin. Alpha cyclodextrin consists of a ring of 6 glucose units linked through 1,4-α-glycosidic bonds. Beta cyclodextrin is a ring of seven glucose units while gamma is a ring of eight glucose units.

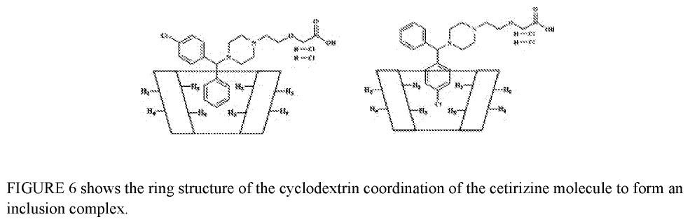

Alpha cyclodextrin consists of a ring of 6 glucose units while beta has 7 glucose units in a ring and gamma has 8 glucose units in a ring. The ring structures form a crown. The inside of the crown is able to complex with APIs in the current application. FIG. 6 shows the process of complexing with cetirizine molecule. Either the phenyl group or the 4-chlorophenyl group fit inside the ring structure. The inside cavity of cylcodextrins is largely hydrophobic, which is favorable for APIs' hydrophobic aromatic systems. The formation of the chelate structure is endothermically favorable due to electrostatic interactions of the pi system of the aromatic moiety with in the hydrophobic cavity and electronic interaction with the hydrogen atoms and glycidyl ether bonds. It is these electronic interactions between these systems of the cyclodextrin and the pi system that gives the favorable heat of formation. The alpha, beta, and gamma cyclodextrins do not form the complex with cetirizine equally. Cetirizine forms more stable complex with β-cyclodextrin (K(a)=5641±358 M(−1)) than α-cyclodextrin (K(a)=1434±60 M(−1)). The association constants from ITC measurements for cetirizine-γ-cyclodextrin and cetirizine-α-cyclodextrin complexes were found to be 1200±50 and 1434±60 (−1) while the cetirizine-β-cyclodextrin complex was 5641±358 M(−1). The beta cyclodextrin has nearly four times the formation constant as the alpha and gamma cyclodextrin. In one embodiment, the complexing composition consists essentially of beta-cyclodextrin.

FIG. 6 shows the ring structure of the cyclodextrin coordination of the cetirizine molecule to form an inclusion complexe. The interior of the cylcodextrin is able to electronically interact with the phenyl groups of the cetirizine molecule. The phenyl group is a reverse quadrapole where the interior of the aromtic ring is very high in electron density and the exterior of the ring is electron deficent. The hydrogen atoms of the hydrophobic interior of the cyclodextrin is electronically attracted to the pi system of the aromatic ring. The hydrogen atoms of the aromatic ring are electronically attracted to the oxygen atoms of the cyclodextrin.

Diphenhydramine forms complexes with cylcodextrins. The mechanism is similar to that of cetirizine. Diphenhydramine forms the more stable complex with β-cyclodextrin (K(a)=4988 M(−1)) than either α-cyclodextrin and γ-cyclodextrin which are both K(a)=1000 M(−1). In one embodiment, the complexing agent comprises beta cyclodextrin, which is configured to form antihistamine complex. In some embodiments, the complexing agent comprises alpha-cyclodextrin, gamma-cyclodextrin, or a combination there. The action of coordination with diphenhydramine is similar to that of cetirizine.

Cluster dextrin also coordination with cetirizine or diphenhydramine. Cluster dextrins have a broad range of cyclic ring and helical structures. Statistically some cyclic and helical structures meet the criteria for complexing with cetirizine or diphenhydramine. Without being limited by theory, the mechanism of complexing between cetirizine or diphenhydramine and cluster dextrin molecules is the same electronic interactions that occur as the alpha, beta, and gamma cyclodextrins.

The gummy pharmaceutical composition may further include flavoring modulating agent. In one embodiment, the flavor modulating agent comprises mannitol. Mannitol is a sugar-alcohol. It is derived from the aldose called mannose. Mannitol can help mask bitterness. Mannitol masks bitterness by a mechanism that involves the endothermic nature of mannitol dissolving into water. In one embodiment, the flavoring modulating agent comprises taurine. Taurine, or 2-aminoethanesulfonic acid, is an organic compound that is widely distributed in animal tissues. Taurine can reduce bitterness by 50% when added at a concentration of 300 mM.

The application further provides methods for making a pharmaceutical gummy composition containing antihistamine. In one embodiment, the pharmaceutical composition may be a low glycemic composition having a glycemic index of less than 8, 10, 15, 20 or 25. In one embodiment, the pharmaceutical composition may be a sugar free gummy composition.

The application further provides methods for using a pharmaceutical gummy composition containing antihistamine for treating allergy.

### EXAMPLES

### Example 1. Cetirizine Gelatin Gummy

Ingredients: Sucrose, Glucose Syrup, 1.3 g Cetirizine HCl, Water, Potassium Sorbate, Sodium Benzoate, Gelatin, Mannitol, Taurine

Gelatin, mannitol, cetirizine and taurine were shifted together. The dry component mixture was added to water to provide a rubbery mass. The rubbery mass was heated at 160° F. until free of foam and a clear yellow. Water was mixed with potassium sorbate, sodium benzoate, glucose and sucrose. The solution was heated until 248° F. The solution was then cooled to 200° F. and the gelatin solution was added. The mixture was stirred until homogenous. The solution was then added to silicone molds and the molds were placed in the refrigerator for 90 minutes. The gummy pieces were removed from the molds to yield products containing roughly 11 mg cetirizine per piece.

### Example 2. Cetirizine Gummy

Performed as stated in Example 1, but with replacing sucrose with palatinose.

### Example 3. Cetirizine Gummy with a Low Glycemic Index Gummy Base

Performed as stated in Example 1, but with replacing sucrose with palatinose and substituting psicose syrup for glucose syrup.

### Example 4. Cetirizine Pectin Gummy

Ingredients: Water (Heated to 200° F.), Sucrose, Pectin Mix (dextrose, pectin, citric acid), Sodium Citrate, Potassium Citrate, Boiling Glucose Syrup, Cetirizine Hydrochloride, 50% Citric Acid in Water, Orange Extract, Orange Color

Sucrose, pectin mixture, sodium citrate and potassium citrate were combined in a separate container to create Mix 1. Cetirizine hydrochloride, 50% citric acid solution, orange extract and orange color were combined in a separate container to create Mix 2. Mix 1 was added to the hot water and the solution was brought to a boil. Then a boiling glucose syrup was added and heated until Brix 83. Mix 2 was then added. The total mixture was stirred until homogeneous. The liquid gummy mix was then added to silicone molds. The gummy pieces were removed from the molds to yield products containing 11 mg cetirizine per piece.

### Example 5. Cetirizine Gummy

Same as example 4 but with substitution of trehalose for sucrose.

### Example 6. Cetirizine Gummy with a Low Glycemic Index

Sample as example 4 but with substitution of trehalose for sucrose and replacing glucose syrup with tagatose syrup.

### Example 7. Cetirizine Gummy with a Low Glycemic Index

Same as example 4 but with replacing sucrose with a mix of palatinose and trehalose.

### Example 8. Cetirizine Gummy with a Low Glycemic Index

Same as example 4 but with substituting a mix of palatinose and trehalose for sucrose and replacing glucose syrup with psicose syrup.

### Example 9. Cetirizine Gummy with a Low Glycemic Index

Same as example 4 but with substituting a mix of palatinose and trehalose for sucrose and replacing glucose syrup with tagatose syrup.

### Example 10. Cetirizine Gelatin Ginger Gummy

Ingredients: Sucrose, Ginger Juice Concentrate, Glucose Syrup, Cetirizine Hydrochloride, Gelatin, Mannitol, Taurine, Sodium Benzoate, Potassium Sorbate

Gelatin, cetirizine and mannitol were shifted together. The dry component mixture was added to water with dissolved potassium sorbate and sodium benzoate to provide a rubbery mass. The rubbery mass was heated at 160° F. until free of foam and a clear yellow. Ginger juice concentrate was added to a container with sucrose and glucose syrup. The mixture was heated until Brix level of 87.5 was reached. The mixture was cooled to 200° F. and the gelatin mix was added with stirring. The final Brix was 84. The solution was then added to silicone molds and the molds were placed in the refrigerator. The gummy pieces were removed from the molds to yield products containing 11 mg cetirizine per piece.

### Example 11. Cetirizine Pectin Gummy w/Fructose

Ingredients: Water, Sucrose, Fructose, Custer dextrin, Pectin Mix (dextrose, pectin, citric acid), Sodium Citrate, Potassium Citrate, (Boiling) Glucose Syrup, Cetirizine Hydrochloride, 50% Citric Acid in Water, Orange Natural Flavor, Orange Color

Pectin mixture, sodium citrate and potassium citrate were combined to create Mix 1. Sucrose, fructose, and cluster dextrin were combined to create Mix 2. Cetirizine hydrochloride, 50% citric acid solution, orange extract and orange color were combined to create Mix 3. Mix 1 was added to the hot water and brought to a boil. The pectin was allowed to fully swell. Mix 2 was then added followed by addition of boiling glucose syrup. The system was heated until Brix 83. Mix 3 was added to provide a gummy batter. The gummy batter was then added to silicone molds. The gummy pieces were removed from the molds to yield products containing 11 mg cetirizine per piece.

### Example 12. Cetirizine Pectin Gummy

Ingredients: Water, citrus pectin, Sucrose, Sodium Citrate, Sucrose, Corn Syrup, Blood Orange Extract, Cetirizine HCl, 50% Citric Acid solution (in 50% glycerol/water), glycerol, orange color

Pectin, sucrose, and the sodium citrate were mixed together. The components are mixed until homogeneous to provide Mix 1. In a separate container is added the citric acid solution, orange color, blood orange flavor and the cetirizine. All is mixed and warmed to 175° F. to provide Mix 2. Water was heated to 200° F. and mixed with Mix 1. The mixture is stirred until the pectin fully swells. The solution is brought to a boil. To the boiling Mix 1 solution was added boiling Corn Syrup. The mixture was heated to Brix 82 at which time Mix 2 was added. The gummy batter was then added to silicone molds. The gummy pieces were removed from the molds to yield products containing 11 mg cetirizine per piece.

### Example 13. Cetirizine Pectin Gummy

Ingredients: Water, pectin, Sucrose, Sodium Citrate, Sucrose, Clusterdextrin, Mannitol, Coconut Oil, (Boling) Glucose Syrup, Blood Orange Extract, Cetirizine HCl, 50% Citric Acid solution (in 50% glycerol/water), glycerol, orange color

Pectin, sucrose, and the sodium citrate were mixed until homogeneous to provide Mix 1. Sucrose, cluster dextrin, and mannitol were mixed to provide Mix 2. Citric acid solution, orange color, blood orange flavor and the cetirizine were mixed and warmed to 175° F. to provide Mix 3. Water is heated to 200° F. and mixed with Mix 1. To the boiling Mix 1 solution was added Mix 2. Coconut oil is then added dropwise to the boiling mixture with stirring. The boiling glucose syrup is then added to the boiling pectin/sugars/oil mix. The mixture was heated to Brix 82 at which time Mix 3 was added dropwise with stirring. The gummy batter was then added to silicone molds. The gummy pieces were removed from the molds to yield products containing 11 mg cetirizine per piece.

### Example 14. Cetirizine Pectin Gummy

As in Example 12 but replacing cluster dextrin with 5.0 grams of alpha-cyclodextrin.

### Example 15. Cetirizine Pectin Gummy

As in Example 12 but replacing cluster dextrin with 10.0 grams of alpha-cyclodextrin.

### Example 16. Cetirizine Pectin Gummy

As in Example 12 but replacing cluster dextrin with 15.0 grams of alpha-cyclodextrin.

### Example 17. Cetirizine Pectin Gummy

As in Example 12 but replacing cluster dextrin with 5.0 grams of beta-cyclodextrin.

### Example 18. Cetirizine Pectin Gummy

As in Example 12 but replacing cluster dextrin with 10.0 grams of beta-cyclodextrin.

### Example 19. Cetirizine Pectin Gummy

As in Example 12 but replacing cluster dextrin with 15.0 grams of beta-cyclodextrin.

### Example 20. Cetirizine Pectin Gummy

As in Example 12 but replacing cluster dextrin with 5.0 grams of gamma-cyclodextrin.

### Example 21. Cetirizine Pectin Gummy

As in Example 12 but replacing cluster dextrin with 10.0 grams of gamma-cyclodextrin.

### Example 22. Cetirizine Pectin Gummy

As in Example 12 but replacing cluster dextrin with 15.0 grams of gamma-cyclodextrin.

### Example 23. Cetirizine Gummy with Low Glycemic Index Gummy Base

Ingredients: Water, pectin (Herbstreith & Fox), Trehalose, Sodium Citrate, Palatinose, Clusterdextrin, Mannitol, Coconut Oil, (Boling) Psicose Syrup, Blood Orange Extract, Cetirizine HCl, 50% Citric Acid solution (in 50% glycerol/water), glycerol, orange color

Pectin, trehalose, and the sodium citrate were mixed to provide mix 1. In a separate container, trehalose, palatinose, cluster dextrin, and mannitol were mixed to provide mix 2. Separately, the citric acid solution, orange color, blood orange flavor and the cetirizine were mixed and warmed to 175° F. until to provide Mix 3.

Water is heated to 200° F. and mixed with Mix 1. The mixture is stirred until the pectin fully swells. The solution is brought to a boil. To the boiling Mix 1 solution was added Mix 2. Coconut oil was then added to the boiling mixture with stirring. The boiling glucose syrup was then added to the boiling mix. The mixture was heated to Brix 82 at which time Mix 3 was added to provide a gummy batter. The gummy batter was then added to silicone molds. The gummy pieces were removed from the molds to yield products containing 11 mg cetirizine per piece.

### Example 24. Cetirizine Gummy with Low Glycemic Index Gummy Base

As in Example 22 but replacing cluster dextrin with 5.0 grams of alpha-cyclodextrin.

### Example 25. Cetirizine Gummy with Low Glycemic Index Gummy Base

As in Example 22 but replacing cluster dextrin with 10.0 grams of alpha-cyclodextrin.

### Example 26. Cetirizine Gummy with Low Glycemic Index Gummy Base

As in Example 22 but replacing cluster dextrin with 15.0 grams of alpha-cyclodextrin.

### Example 27. Cetirizine Gummy with Low Glycemic Index Gummy Base

As in Example 22 but replacing cluster dextrin with 5.0 grams of beta-cyclodextrin.

### Example 28. Cetirizine Gummy with Low Glycemic Index Gummy Base

As in Example 22 but replacing cluster dextrin with 10.0 grams of beta-cyclodextrin.

### Example 29. Cetirizine Gummy with Low Glycemic Index Gummy Base

As in Example 22 but replacing cluster dextrin with 15.0 grams of beta-cyclodextrin.

### Example 30. Cetirizine Gummy with Low Glycemic Index Gummy Base

As in Example 22 but replacing cluster dextrin with 5.0 grams of gamma-cyclodextrin.

### Example 31. Cetirizine Gummy with Low Glycemic Index Gummy Base

As in Example 22 but replacing cluster dextrin with 10.0 grams of gamma-cyclodextrin.

### Example 32. Cetirizine Gummy with Low Glycemic Index Gummy Base

As in Example 22 but replacing cluster dextrin with 15.0 grams of gamma-cyclodextrin.

### Example 33. Agar Cetirizine Gummy

Ingredients: Water, Ticagel Natural GC-581 B, Corn Syrup, Sucrose, Cetirizine HCl, Citric Acid 50% in Water, orange color, Orange Natural Flavor, Glycerine

Mix Ticagel with water at 175° F. to provide an agar mix. Corn syrup that was prewarmed to 175° F. was combined with the agar mix. Sucrose with then added and the mixture was heated until a Brix of 80 was reached. The 50% citric acid, color, orange natural flavor, cetirizine, and glycerol were combined until homogenous. This mixture was then added to the batter. The batter was then deposited into silicone molds and cured at 98° F. and 18% relative humidity for 24 hours. The gummy pieces were removed from the molds to yield products containing 11 mg cetirizine per piece.

### Example 34. Kappa Carrageenan Cetirizine Gummy

Ingredients: Water, Kappa Carrageenan, Potassium Citrate, Sucrose, Coconut oil, Sucrose, Glucose Syrup, Cetirizine HCl, Mannitol, Blood Orange Extract

Kappa carrageenan, sucrose, and potassium citrate were mixed together with water and heated 200° F. until lump free. In a separate container was added the glucose syrup with 220 grams of sucrose and the mannitol. These were heated until Brix 88 was reached. The mixture was cooled to 210° F. and the above solution of kappa carrageenan was added. Cetirizine, blood orange extract, and 5 grams water were combined and added to the kappa/sugar mixture. The resulting gummy batter was then added to silicone molds. The gummy pieces were removed from the molds to yield products containing 11 mg cetirizine per piece.

### Example 35. Liquid Chromatography Mass Spectrometry of Example 12

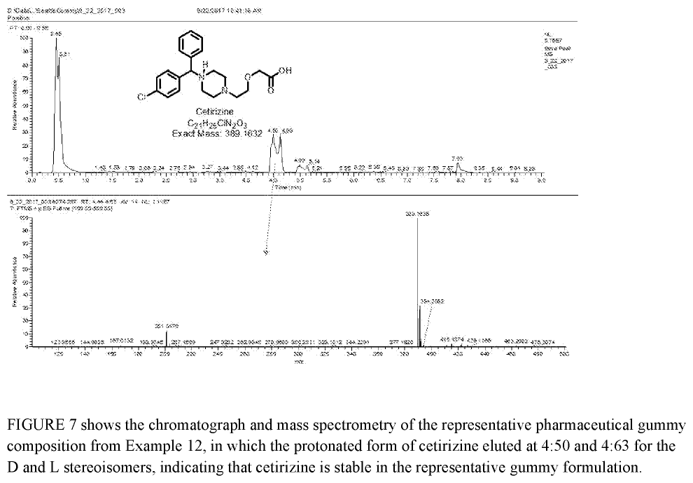

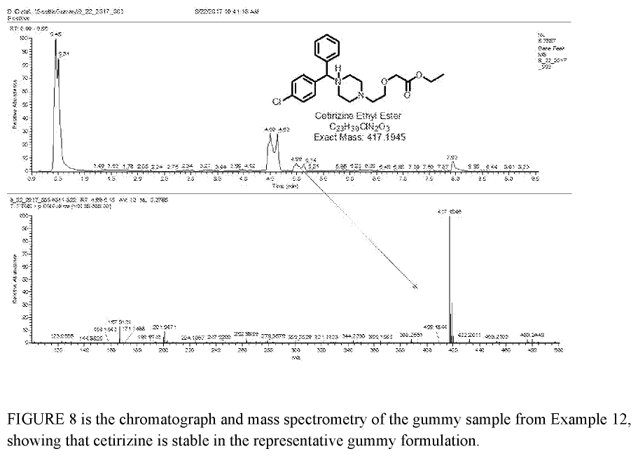

A 7.5-gram piece of gummy from Example 13 was added to 150 mL PBS in a sealed container. The PBS container was placed in a water bath heated to 99° F. and the water bath was placed on a shaker table. The shaker table was turned to half speed and the gummy was allowed to dissolve. Full dissolving took about 90 minutes. The resulting solution was placed on a LTQ-Orbitrap for analysis. The protonated form of cetirizine eluted at 4:50 and 4:63 for the D and L isomers. The protonated form of cetirizine ethyl ester eluted at eluted at 4:99 and 5:14 for the D and L isomers. These are shown in FIGS. 7 and 8. The results show that the cetirizine is in stable in the gummy formulation. The gummy pieces were removed from the molds to yield products containing 11 mg cetirizine per piece.

### Example 36. Liquid Chromatography Mass Spectrometry of Example 29

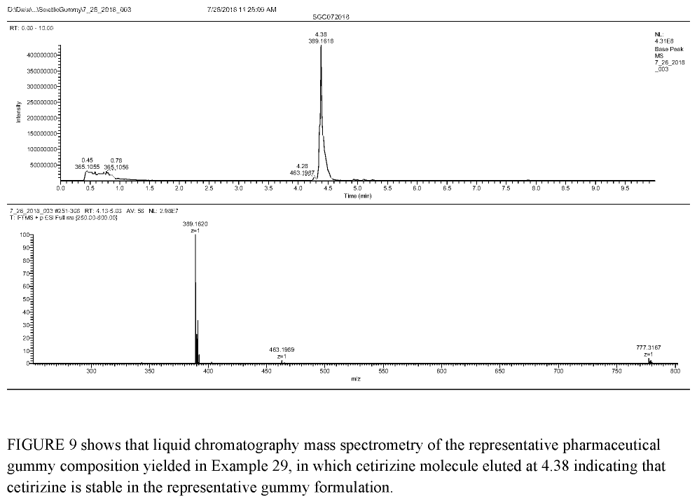

A 7.5-gram piece of gummy from Example 29 was added to 150 mL PBS in a sealed container. The PBS container was placed in a water bath heated to 99° F. and the water bath was placed on a shaker table. The shaker table was turned to half speed and the gummy was allowed to dissolve. Full dissolving took about 90 minutes. The resulting solution was placed on an LTQ-Orbitrap for analysis. The protonated form of cetirizine eluted at 4:38 as shown in FIG. 9. The results show that the cetirizine is in the gummy sample and that the cetirizine molecule survived the manufacturing process. The gummy pieces were removed from the molds to yield products containing 11 mg cetirizine per piece.

### Example 37. Diphenhydramine HCl Gummy

Ingredients: 2.2 g Diphenhydramine HCl, Sodium Benzoate, Potassium Sorbate, 240.0 g Sucrose, 283.0 g Glucose Syrup, Water, Gelatin, Sorbitol, organic ground Ginger

Gelatin and sorbitol were mixed and was added to water to provide a rubbery mass. The rubbery mass was heated at 160° F. until free of foam and a clear yellow.

245 grams of water was mixed with diphenhydramine HCl at a boiling temperature and followed by addition of potassium sorbate and sodium benzoate to provide the boiling diphenhydramine HCl solution. Glucose syrup was added. Sucrose and ginger were shifted together and the mix was added to the diphenhydramine HCl/glucose solution. The solution was heated until 248° F. was reached. The solution was then cooled to 200° F. The gelatin solution was added. The solution was then added to silicone molds and the molds were placed in the refrigerator for 90 minutes. The result was chewy gummy product with 25 mg diphenhydramine HCl per 7.5-gram gummy with ginger spice flavor.

### Example 38. Diphenhydramine HCl Gummy

Ingredients: 240.0 g Sucrose, 283.0 g Glucose Syrup, 2.390 g Diphenhydramine Hydrochloride, Gelatin, Sorbitol, Water, Sodium Benzoate, Potassium Sorbate, Watermelon Flavoring, Coconut Flavoring

Gelatin and sorbitol were shifted together and the mixture was added to water with dissolved potassium sorbate and sodium benzoate to provide a rubbery mass. The rubbery mass was heated at 160° F. until free of foam and a clear yellow. Diphenhydramine was dissolved in water and was added glucose syrup with stirring. The solution was then brought to a boil. The sucrose mix was then added to the boiling solution. The solution was heated until a brix level of 85 was reached. The solution was then cooled to 200° F. The gelatin solution was added. The mixture was stirred until homogenous. Then the watermelon and coconut flavorings were added. The solution was then added to silicone molds and the molds were placed in the refrigerator for 90 minutes. Each 7.5-gram gummy had roughly 25 mg diphenhydramine.

### Example 39. Diphenhydramine Gummy

Ingredients: 240.0 g Sucrose, 200.1 g Ginger Juice Concentrate, 100.0 g Glucose Syrup, 2.388 g Diphenhydramine Hydrochloride, 62.5 g Gelatin, 18.8 g Sorbitol, Sodium Benzoate, Potassium Sorbate

Gelatin and sorbitol were shifted together mixed with water with dissolved potassium sorbate and sodium benzoate to provide a rubbery mass. The rubbery mass was heated at 160° F. until free of foam and a clear yellow. Ginger juice concentrate was mixed with sucrose and glucose syrup. The mixture was heated until a Brix level of 87.5 was reached. The gelatin mix was added. The temperature was maintained to remain above 180° F. A mix of 2.388 g of diphenhydramine in 4.035 g water was then added dropwise over a period of 30 seconds with stirring. The solution was then added to silicone molds and the molds were placed in the refrigerator for 90 minutes. Each 7.5-gram gummy had roughly 25 mg of diphenhydramine.

### Example 40. Diphenhydramine Gummy

Ingredients: 240.3 g Sucrose, 200.1 g Ginger Juice Concentrate, 100.5 g Glucose Syrup, 2.350 g Diphenhydramine Hydrochloride, 62.5 g Gelatin, 18.8 g Mannitol, 21.2 g Taurine, Sodium Benzoate, Potassium Sorbate

Gelatin and mannitol were mixed with water with dissolved potassium sorbate and sodium benzoate to provide a rubbery mass. The rubbery mass was heated at 160° F. until free of foam and a clear yellow. Ginger juice concentrate was mixed sucrose and glucose syrup. The mixture was heated until a Brix level of 87.5 was reached. The gelatin mix was added followed by the addition of a solution of 2.350 g of diphenhydramine hydrochloride in 6.100 g water. The final Brix was 84. The resulting mixture was then added to silicone molds and the molds were placed in the refrigerator for 90 minutes. Each 7.5-gram gummy had roughly 20 mg of diphenhydramine.

### Example 41. Diphenhydramine Hydrochloride Containing Pectin Gummy

Ingredients: 5.500 grams Sodium Bicarbonate, 3.673 grams Potassium Hydroxide food grade, 1.555 grams Diphenhydramine Hydrochloride, 145.3 grams Citrus Pectin, Water, 409.1 grams Glucose Syrup, 310.3 grams Sucrose, 15.0 grams 50% citric acid in water, Cady Apple Green Green Food Color

Mix water, potassium hydroxide and sodium bicarbonate to provide a basic solution. Add the pectin. Stir until homogenous. The glucose syrup was then added. The sucrose was then added and the total mixture was heated until Brix 84.

The mixture was cooled to 210° F. and a mixture of citric acid solution, candy apple green flavor and diphenhydramine hydrochloride was added to provide a batter mixture. Green food color was then added. The gummy batter was then added to silicone molds and allowed to cool to room temperature. Excellent tasting gummies resulted. Each 7.5-gram gummy had roughly 13 mg diphenhydramine hydrochloride.

### Example 42. Diphenylhydramine HCl Lemon Gummy

Ingredient: Water, Pectin, 100 grams Fructose, 3.0 grams Sodium Citrate, 200.0 grams Sucrose, 20.0 grams Mannitol, 20.0 grams β-Cyclodextrin, 410.0 grams Glucose Syrup, 6.0 grams Coconut Oil, 25.0 grams 50% Citric Acid Solution (50% glycerol/water), Turmeric Natural Yellow, 1.426 grams Diphenhydramine hydrochloride, Natural Lemon Flavor

Water is added to a container and heated to 200° F. To a separate container were added pectin, fructose, and sodium citrate to provide Mix 1. To a separate container were added sucrose, 15 g cyclodextrin, and mannitol to provide Mix 2. To a separate container were mixed the citric acid solution, diphenhydramine, 5 g cyclodextrin and the yellow colorant to provide Mix 3. Mix 1 was added to the heated water and allowed to dissolve. Mix 2 was then added.

In a separate container was added glucose syrup and the coconut oil. This solution was heated until Brix 90-93 has been reached. The temperature is ˜245° F. The glucose syrup is then added. The solution was heated until Brix 80 has been reached. The lemon flavor was added. In a separate container, Mix 3 was prewarmed to 175° F. and then added to the gummy batter solution. The batter was stirred until homogenous and then poured into silicone molds. The molds were then allowed to cool to room temperature. Each 7.5-gram gummy had 12 mg of diphenhydramine hydrochloride.

### Example 43. Diphenhydramine Watermelon Gummy

Ingredients: Water, Pectin, 100 grams Fructose, 3.0 grams Sodium Citrate, 220.0 grams Sucrose, 20.0 grams Mannitol, 20.2 grams β-Cyclodextrin, 410.0 grams Glucose Syrup, 25.0 grams 50% Citric Acid Solution (50% glycerol/water), Carmine Red, 1.426 grams Diphenhydramine hydrochloride, Natural Watermelon Flavor

Water is added to a container and heated to 200° F. To a separate container were mixed pectin, fructose, and sodium citrate to provide Mix 1. To a separate container were mixed sucrose, 15 g cyclodextrin, and mannitol to provide Mix 2. To a separate container were mixed the citric acid solution, diphenhydramine, 5 g cyclodextrin and the red colorant to provide Mix 3. Mix 1 was added to the heated water and allowed to dissolve. With continued stirring, Mix 2 as added. The resulting mixture was mixed until all components were dissolved.

In a separate container was added glucose syrup and heated until Brix 90-93 has been reached. The temperature is ˜245° F. The solution is heated until Brix 81 has been reached. Then the lemon flavor was added. In a separate container, Mix 3 was prewarmed to 175° F. Mix 3 was then added dropwise to the gummy batter solution with string. The batter was stirred until homogenous and then poured into silicone molds. The molds were then allowed to cool to room temperature. Each 7.5-gram gummy had 11.5 mg of diphenhydramine hydrochloride.

### Example 44. Diphenhydramine Watermelon Gummy

Ingredients: Water, 105.0 grams Pectin, 300 grams Fructose, 6.0 grams Sodium Citrate, 660.0 grams Sucrose, 30.2 grams β-Cyclodextrin, 25 g Glycerol, 1230.0 grams Glucose Syrup, 60.0 grams 50% Citric Acid Solution (50% glycerol/water), Carmine Red, 9.2 grams Diphenhydramine hydrochloride, Natural Watermelon Flavor

Water was added to a container and heated to 200° F. Diphenhydramine hydrochloride was allowed to dissolve. To a separate container were mixed pectin, fructose, cyclodextrin and sodium citrate to provide Mix 1. To a separate container are added sucrose and 15 g cyclodextrin to provide Mix 2. To a separate container were mixed the citric acid solution, watermelon flavor and the red colorant to provide Mix 3.

With stirring, Mix 1 was added to the heated water and allowed to dissolve. In a separate container was added glucose syrup is brought to a boil. To the boiling glucose syrup was added Mix 2 followed by the remaining water. The sugars were allowed to disperse and the solution heated to near the boiling point. The dissolved pectin solution is then slowly added to the glycose syrup solution. The solution was heated and boiled until Brix 83 has been reached at which point the system is cooled to 210-210° F.

In a separate container, Mix 3 was prewarmed to 175° F. Mix 3 was then added dropwise to the gummy batter solution with string. The batter was stirred until homogenous and then poured into silicone molds. The molds were then allowed to cool to room temperature. Each 3.75-gram gummy had 12.5 mg of diphenhydramine hydrochloride.

### Example 45. Liquid Chromatography Mass Spectrometry of Example 44

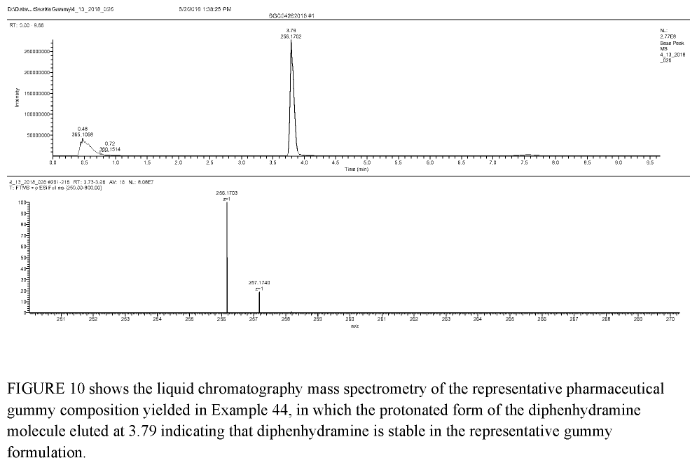

A 3.75-gram piece of gummy from Example 44 was added to 150 mL PBS in a sealed container. The PBS container was placed in a water bath heated to 99° F. and the water bath was placed on a shaker table. The shaker table was turned to half speed and the gummy was allowed to dissolve. Full dissolving took about 45 minutes. The resulting solution was placed on a LTQ-Orbitrap for analysis. The protonated form of diphenhydramine eluted at 3.79. The data is shown in FIG. 10. The results show that the diphenhydramine is in the gummy sample and that the diphenhydramine molecule survived the manufacturing process. The gummy pieces were removed from the molds to yield products containing 12.5 mg diphenhydramine per piece.

### Example 46. Diphenhydramine Orange Gummy

Ingredients: Water, 105.0 grams Pectin, 300 grams Fructose, 6.0 grams Sodium Citrate, 846.0 grams Sucrose, 30.2 grams β-Cyclodextrin, 20.0 grams Mannitol, Glycerol, 1000.0 grams Glucose Syrup, 60.0 grams 50% Citric Acid Solution (50% glycerol/water), Paprika Orange, 9.4 grams Diphenhydramine hydrochloride, Natural Orange Flavor

Water is added to a container and heated to 200° F. To the water was added the diphenhydramine hydrochloride and allowed to dissolve. To a separate container were added pectin, fructose, 15 g of cyclodextrin and sodium citrate to provide Mix 1. To a separate container were added sucrose, mannitol and 15 g cyclodextrin to provide Mix 2. To a separate container were mixed the citric acid solution, flavor and the orange colorant to provide Mix 3.

Mix 1 was added to the heated water and allowed to dissolve. In a separate container was added glucose syrup and brought to a boil. To the boiling glucose syrup was added Mix 2 followed by water. The sugars were allowed to disperse and the solution heated to near the boiling point. The dissolved pectin solution was then slowly added to the glycose syrup solution. The solution is heated and boiled until Brix 83 has been reached at which point the system is cooled to 210-210° F. In a separate container, Mix 3 was prewarmed to 175° F. Mix 3 was then added to the gummy batter solution with string. The batter was stirred until homogenous and then poured into silicone molds. The molds were then allowed to cool to room temperature. Each 3.75-gram gummy had 12.5 mg of diphenhydramine hydrochloride.

### Example 47. Diphenhydramine Orange Gummy

As in Example 46 but replacing beta-cyclodextrin with 15.0 grams of alpha-cyclodextrin.

### Example 48. Diphenhydramine Orange Gummy

As in Example 46 but replacing beta-cyclodextrin with 20.0 grams of alpha-cyclodextrin.

### Example 49. Diphenhydramine Orange Gummy

As in Example 46 but replacing beta-cyclodextrin with 30.0 grams of alpha-cyclodextrin.

### Example 50. Diphenhydramine Orange Gummy

As in Example 46 but replacing beta-cyclodextrin with 15.0 grams of beta-cyclodextrin.

### Example 51. Diphenhydramine Orange Gummy

As in Example 46 but replacing beta-cyclodextrin with 20.0 grams of beta-cyclodextrin.

### Example 52. Diphenhydramine Orange Gummy

As in Example 46 but replacing beta-cyclodextrin with 15.0 grams of gamma-cyclodextrin.

### Example 53. Diphenhydramine Orange Gummy

As in Example 46 but replacing beta-cyclodextrin with 20.0 grams of gamma-cyclodextrin.

### Example 54. Diphenhydramine Orange Gummy

As in Example 46 but replacing beta-cyclodextrin with 30.0 grams of gamma-cyclodextrin.

### Example 55. Diphenhydramine Orange Gummy

Ingredients: Water, Pectin, 300 grams Fructose, Sodium Citrate, 846.0 grams Sucrose, 30.2 grams β-Cyclodextrin, 20.0 grams Mannitol, Glycerol, 1000.0 grams Glucose Syrup, 60.0 grams 50% Citric Acid Solution (50% glycerol/water), Paprika Orange, 9.4 grams Diphenhydramine hydrochloride, Natural Orange Flavor

Water was added to a container and heated to 200° F. To the water was added the diphenhydramine hydrochloride and allowed to dissolve. To a separate container were added pectin, fructose, 15 g of cyclodextrin and sodium citrate to provide Mix 1. To a separate container were added sucrose, mannitol and 15 g cyclodextrin to provide Mix 2. To a separate container are mixed the citric acid solution, flavor and the orange colorant to provide Mix 3.

Mix 1 was added to the heated water and allowed to dissolve. In a separate container was added glucose syrup and was brought to a boil. To the boiling glucose syrup was added Mix 2 followed by the remaining water. The sugars were allowed to disperse and the solution heated to near the boiling point. The dissolved pectin solution was then slowly added to the glycose syrup solution.

The solution was heated and boiled until Brix 83 has been reached at which point the system is cooled to 210-210° F. In a separate container, Mix 3 was prewarmed to 175° F. Mix 3 was then added dropwise to the gummy batter solution with string. The batter was stirred until homogenous and then poured into silicone molds. The molds were then allowed to cool to room temperature. Each 3.75-gram gummy had 12.5 mg of diphenhydramine hydrochloride.

### Example 56. Diphenhydramine Orange Gummy

As in Example 53 but replacing beta-cyclodextrin with 15.0 grams of alpha-cyclodextrin.

### Example 57. Diphenhydramine Orange Gummy

As in Example 53 but replacing beta-cyclodextrin with 20.0 grams of alpha-cyclodextrin.

### Example 58. Diphenhydramine Orange Gummy

As in Example 53 but replacing beta-cyclodextrin with 30.0 grams of alpha-cyclodextrin.

### Example 59. Diphenhydramine Orange Gummy

As in Example 53 but replacing beta-cyclodextrin with 15.0 grams of beta-cyclodextrin.

### Example 60. Diphenhydramine Orange Gummy

As in Example 53 but replacing beta-cyclodextrin with 20.0 grams of beta-cyclodextrin.

### Example 61. Diphenhydramine Orange Gummy

As in Example 53 but replacing beta-cyclodextrin with 15.0 grams of gamma-cyclodextrin.

### Example 62. Diphenhydramine Orange Gummy

As in Example 53 but replacing beta-cyclodextrin with 20.0 grams of gamma-cyclodextrin.

### Example 63. Diphenhydramine Orange Gummy

As in Example 53 but replacing beta-cyclodextrin with 30.0 grams of gamma-cyclodextrin.

### Example 64. Diphenhydramine Orange Gummy that is Safer for Diabetics

Ingredients: Water, Pectin, Isomaltulose, grams Sodium Citrate, grams Trehalose, β-Cyclodextrin, 20.0 grams Mannitol, 25 g Glycerol, grams Psicose, 60.0 grams 50% Citric Acid Solution (50% glycerol/water), Paprika Orange, 9.4 grams Diphenhydramine hydrochloride, Natural Orange Flavor

Water was added to a container and heated to 200° F. To the water was added the diphenhydramine hydrochloride and allowed to dissolve. The trehalose and isomaltulose were combined and mixed until uniform to provide the sugar mixture. To a separate container were added pectin, 300 grams of the sugar mixture, 15 g of cyclodextrin and sodium citrate to provide Mix 1. To a separate container were added the remaining sugar mixture, mannitol and 15 g cyclodextrin to provide Mix 2. To a separate container are mixed the citric acid solution, flavor and the orange colorant to provide Mix 3.

Mix 1 was added to the heated water and allowed to dissolve. In a separate container was added psicose and water brought to a boil. To the boiling psicose syrup was added Mix 2 followed by water. The sugars were allowed to disperse and the solution heated to near the boiling point. The dissolved pectin solution was then added to the glycose syrup solution.

The solution is heated and boiled until Brix 83 has been reached at which point the system is cooled to 210-210° F. In a separate container, Mix 3 was prewarmed to 175° F. Mix 3 was then added dropwise to the gummy batter solution with string. The batter was stirred until homogenous and then poured into silicone molds. The molds were then allowed to cool to room temperature. Each 3.75-gram gummy had 12.5 mg of diphenhydramine hydrochloride.

### Example 65. Diphenhydramine Orange Gummy that is Safer for Diabetics

As in Example 64 but replacing beta-cyclodextrin with 15.0 grams of alpha-cyclodextrin.

### Example 66. Diphenhydramine Orange Gummy that is Safer for Diabetics

As in Example 64 but replacing beta-cyclodextrin with 20.0 grams of alpha-cyclodextrin.

### Example 67. Diphenhydramine Orange Gummy that is Safer for Diabetics

As in Example 64 but replacing beta-cyclodextrin with 30.0 grams of alpha-cyclodextrin.

### Example 68. Diphenhydramine Orange Gummy that is Safer for Diabetics

As in Example 64 but replacing beta-cyclodextrin with 15.0 grams of beta-cyclodextrin.

### Example 69. Diphenhydramine Orange Gummy that is Safer for Diabetics

As in Example 64 but replacing beta-cyclodextrin with 20.0 grams of beta-cyclodextrin.

### Example 70. Diphenhydramine Orange Gummy that is Safer for Diabetics

As in Example 64 but replacing beta-cyclodextrin with 15.0 grams of gamma-cyclodextrin.

### Example 71. Diphenhydramine Orange Gummy that is Safer for Diabetics

As in Example 64 but replacing beta-cyclodextrin with 20.0 grams of gamma-cyclodextrin.

### Example 72. Diphenhydramine Orange Gummy that is Safer for Diabetics

As in Example 64 but replacing beta-cyclodextrin with 30.0 grams of gamma-cyclodextrin.

### Example 73. Diphenhydramine Orange Gummy that is Safer for Diabetics

As in Example 64 but replacing psicose with tagatose.

### Example 72. Diphenhydramine Orange Gummy that is Safer for Diabetics

As in Example 64 but replacing psicose with sorbose.

### Example 73. Diphenhydramine Orange Gummy that is Safer for Diabetics

As in Example 64 but replacing psicose with a tagatose-psicose mixture.

### Example 74. Diphenhydramine Orange Gummy that is Safer for Diabetics

As in Example 64 but replacing psicose with a sorbose-psicose mixture.

### Example 75. Diphenhydramine Orange Gummy that is Safer for Diabetics

As in Example 64 but replacing psicose with a sorbose-tagatose mixture.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Suitable dosage amounts and dosing regimens may be selected in accordance with a variety of factors, including one or more particular conditions being treated, the severity of the one or more conditions, the genetic profile, age, health, sex, diet, and weight of the subject, the route of administration alone or in combination with pharmacological considerations including the activity, efficacy, bioavailability, pharmacokinetic, and toxicological profiles of the particular compound employed, whether a drug delivery system is utilized and whether the drug is administered as part of a drug combination. Therefore, the dosage regimen to be employed may vary widely and may necessarily deviate from the dosage regimens set forth herein.

A dosage form may be administered to a subject in need thereof once per day, or twice per day, or once every 6 hours, or once every 4 hours, or once every 2 hours, or hourly, or twice an hour, or twice a day, or twice a week, or monthly.

The phrase “therapeutically effective” is intended to qualify the amount that will achieve the goal of improvement in disease severity and/or the frequency of incidence over non-treatment, while limiting, reducing, or avoiding adverse side effects typically associated with disease therapies.

The term “pharmaceutically acceptable” is used herein to mean that the modified noun is appropriate for use in a pharmaceutical product. Pharmaceutically acceptable cations include metallic ions and organic ions. Other metallic ions include, but are not limited to appropriate alkali metal salts, alkaline earth metal salts and other physiological acceptable metal ions. Exemplary ions include aluminium, calcium, lithium, magnesium, potassium, sodium and zinc in their usual valences. Organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

It is further contemplated that one active ingredient may be in an extended release form, while an optional second, third, or fourth other active ingredient, for example, may or may not be, so the recipient experiences, for example, a spike in the second, third, or fourth active ingredient that dissipates rapidly, while the first active ingredient is maintained in a higher concentration in the blood stream over a longer period of time. Similarly, one of the active ingredients may be an active metabolite, while another may be in an unmetabolized state, such that the active metabolite has an immediate effect upon administration to a subject whereas the unmetabolized active ingredient administered in a single dosage form may need to be metabolized before taking effect in the subject.

The pharmaceutical preparations may be in unit dosage forms. In such form, the preparation may be subdivided into unit doses containing appropriate quantities of the active component. The unit dosage form can be a packaged preparation, such as a kit or other form, the package containing discrete quantities of preparation, such as packeted tablets, capsules, liquids or powders in vials or ampoules. Also, the unit dosage form can be a capsule, tablet, cachet, or lozenge, or it can be the appropriate number of any of these in packaged form.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A semi-solid pharmaceutical composition, comprising,
a gelling component in a sufficient amount to provide a cohesive gelled product,
an antihistamine composition, comprising an antihistamine, and
a complexing agent, wherein the complexing agent is capable of interacting with the antihistamine and forming an antihistamine complex.

2. The semi-solid pharmaceutical composition of claim 1, wherein the antihistamine comprises acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative thereof.

3. The semi-solid pharmaceutical composition of claim 1, comprising the antihistamine at a concentration not less than about 0.05% w/w.

4. The semi-solid pharmaceutical composition of claim 1, wherein the antihistamine composition further comprises a herbal ingredient, an amino acid, a vitamin, or a mineral having antihistamine or allergy relieving activity.

5. The semi-solid pharmaceutical composition of claim 1, wherein the complexing agent comprises protein, peptide, polyamide, cluster dextrin, cyclodextrin, polydextrose, resistant starch, cellulose, plant particles, calcium carbonate, diatomaceous earth, chitosan, or a combination thereof.

6. The semi-solid pharmaceutical composition of claim 1, wherein the antihistamine composition comprises cetirizine, levocetirizine, diphenhydramine, loratadine, or fexofenadine, and wherein the complexing composition comprising polyamide, cluster dextrin, cyclodextrin, or a combination thereof.

7. The semi-solid pharmaceutical composition of claim 6, wherein the cyclodextrin comprises alpha-dextrin, beta-cyclodextrin, gamma-cyclodextrin, or a combination thereof.

8. The semi-solid pharmaceutical composition of claim 1, wherein the gelling composition comprises gelatin, starch, pectin, gellan gum, gum Arabic, carrageenan, guar, agar, alginate, locust bean gum, xanthan, or derivatives thereof.

9. The semi-solid pharmaceutical composition of claim 1, further comprising a sugar composition, wherein the sugar composition comprises sucrose, glucose, fructose, palatinose, trehalose, psicose, tagatose, sorbose, mannose, maltose or a combination thereof.

10. The semi-solid pharmaceutical composition of claim 9, wherein the sugar composition comprises essentially trehalose, palatinose, psicose, tagatose, sorbose, or a combination thereof.

11. The semi-solid pharmaceutical composition of claim 1, further comprising an herb composition, an antioxidant composition, a vitamin composition, a mineral composition, an amino acid composition, a probiotics composition, or a prebiotics composition.

12. The semi-solid pharmaceutical composition of claim 11, wherein the herb composition comprises butterbur, quercetin, Stinging nettles (Urtica dioica), bromelain, phleum pratense, tinospora cordifolia, European elderflower, sorrel, cowslip, verbena, gentian root, echinacea, grape seed, pycnogenol, pine bark extract, EPA, honey, cat's claw, albizzia (Albizzia lebbeck), baical skullcup (Scutellaria baicalensis), goldenseal, spirulina, bitter orange (citrus aurantium), lemon, eucalyptus, frankincense, Angelica sinensis, eyebright (Euphrasia officinalis), Gingko, milk thistle (Silybum marianum), red clover (Trifolium pratense), Yarrow (Achillea millefolium), rosemary, shiso, sage, peppermint, licorice, Astragalus, Ginseng, Artemisia argyi, Stephania tetrandra, coix seed, Citrus trifoliata, Citrus aurantium, Angelica dahurica, an extract, powder, isolate or distillate thereof.

13. The semi-solid pharmaceutical composition of claim 11, wherein the antioxidant composition comprises Vitamin E, Vitamin C, beta-carotene, gallic acid, selenium, selenium yeast, phenolics, anthocyanins, flavonoids, bioflavonoids, theobromine, anthracenes, carotenoids, lutein, zeaxanthin, ginko biloba, berry extract, resveratrol, saffron, Sangre de grado (dragon's blood), cocoa, or derivatives thereof.

14. The semi-solid pharmaceutical composition of claim 11, wherein the vitamin composition comprises vitamin A, B, C, D, E, K or a combination thereof.

15. The semi-solid pharmaceutical composition of claim 11, wherein the mineral composition comprises salts of calcium, iron, zinc, magnesium, sodium, chloride, potassium, copper, molybdenum, manganese, phosphorus, iodine, nickel, or selenium, or a combination thereof.

16. The semi-solid pharmaceutical composition of claim 11, wherein the mineral composition consists essentially salts of zinc.

17. The semi-solid pharmaceutical composition of claim 11, wherein the amino acid composition comprises histidine, a branched chain amino acid, L-5 hydroxytryptophan (5-HTP), or its derivative thereof.

18. The semi-solid pharmaceutical composition of claim 11, wherein the prebiotic composition comprises gum arabic, chicory root, wheat bran, resistant starch, mannose oligosaccharide, acacia gum, inulin, galacto-oligosaccahride, guar gum, Artichoke fiber, fructo-ligosaccharide, or a combination thereof.

19. The semi-solid pharmaceutical composition of claim 1, further comprising an additive selected from sweeteners, food acids, flavoring agents, coloring agents, humectants, bulking agents, fatty acids, triglycerides, plasticizers, emulsifiers, thickeners, preservatives, or and a mixture thereof.

20. The semi-solid pharmaceutical composition of claim 1, wherein the sweetener comprises erythritol, xylitol, sugar, glucose syrup, corn syrup, high fructose corn syrup, trehalose, isomaltose, psicose, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

21. The semi-solid pharmaceutical composition of claim 1, having a glycemic index of not more than 25.

22. The semi-solid pharmaceutical composition of claim 1, wherein the pH of the composition is about 3 to about 5.

23. A semi-solid pharmaceutical composition, comprising pectin from about 0.5% to about 5% by weight, cetirizine hydrochloride from about 0.05% to about 0.3% by weight, a complexing agent from about 0.1% to about 12% by weight, and a sugar composition from about 60% to about 85% by weight, wherein the complexing agent comprises polyamide, cyclodextrin, or cluster dextrin.

24. The semi-solid pharmaceutical composition of claim 23, wherein the complexing agent comprises essentially cyclodextrin.

25. The semi-solid pharmaceutical composition of claim 23, wherein the sugar composition comprises sucrose, fructose, glucose or a combination thereof.

26. The semi-solid pharmaceutical composition of claim 23, wherein the sugar composition comprises trahalose, palatinose, psicose, or a combination thereof.

27. A semi-solid pharmaceutical composition, comprising pectin from about 0.5% to about 5% by weight, diphenhydramine hydrochloride from about 0.3% to about 1.5% by weight, a complexing agent from about 0.1% to about 12% by weight, and a sugar composition from about 60% to about 85% by weight, wherein the complexing agent comprises polyamide, cyclodextrin, or cluster dextrin.

28. The semi-solid pharmaceutical composition of claim 27, wherein the complexing agent comprises essentially cyclodextrin.

29. The semi-solid pharmaceutical composition of claim 27, wherein the sugar composition comprises sucrose, fructose, glucose or a combination thereof.

30. The semi-solid pharmaceutical composition of claim 27, wherein the sugar composition comprises trehalose, palatinose, psicose, or a combination thereof.
